(19) United States
(12) Patent Application Publication
(10) Pub. No.: US 2007/0212239 A1
(43) Pub. Date: Sep. 13, 2007

(54) VACUUM HAND PUMP AND VACUUM SEALABLE BAG VALVE SYSTEM AND METHOD

(75) Inventors: Ross F. Heil, Walnut Creek, CA (US); Robert A. Wilk, Sierra Village, CA (US); Robert B. Carlsen, Mi-Wuk Village, CA (US)

Correspondence Address: FLIESLER MEYER LLP, 650 CALIFORNIA STREET, 14TH FLOOR, SAN FRANCISCO, CA, 94108, US

(73) Assignee: SIMPLYSMART, LLC, Walnut Creek, CA (US)

(21) Appl. No.: 11/684,336

(22) Filed: Mar. 9, 2007

Related U.S. Application Data
(60) Provisional application No. 60/780,750, filed on Mar. 9, 2006.

(51) Int. Cl. A61M 1/00

(52) U.S. Cl. 417/437

(57) ABSTRACT

A vacuum hand pump and valve system which fits in the palm of a hand and has a deployable crank handle used to turn a vane in order to create a vacuum in a container.

### CLAIM OF PRIORITY

The present invention claims benefit from U.S. Provisional Patent Application No. 60/780,750, filed Mar. 9, 2006, entitled: VACUUM HAND PUMP AND VACUUM SEALABLE BAG WITH BAG VALUE SYSTEM AND METHOD, by Ross Heil, Robert A. Wilk, Robert B. Carlsen, Ph.D. (Attorney Docket No. HEIL-07976US0).

### FIELD OF THE DISCLOSURE

The present invention relates to vacuum packaging equipment and bags for preserving perishable products such as fruits, vegetables, meats, fish and prepared food products, and also for preserving and protecting other materials and things.

### BACKGROUND

It is well known that fruits, vegetable, meats, fish, and prepared food products and other food products can be preserved by removing oxygen from the food packaging environment in order to reduce the oxidation and spoilage of the food. There are a number of products on the market that provide for removing oxygen from storage containers in order to preserve such food products. Additionally products exist that displace the oxygen in a storage environment with another gas such as nitrogen or carbon dioxide in order to reduce oxidation of the food product. Similar technologies have been used to protect other materials and things from exposure to the environment. For example, silver dining ware and jewelry have been stored in such oxygen depleted storage systems in order to for example prevent tarnishing.

### BRIEF DESCRIPTION OF THE DRAWINGS

FIG. 1 is an illustration of a perspective view of a vacuum hand pump assembly of an embodiment of the invention.

FIG. 2 is an illustration of a first side view of the vacuum hand pump assembly of the embodiment of the invention depicted in FIG. 1.

FIG. 3 is an illustration of a second side view of the vacuum hand pump assembly of the embodiment of the invention depicted in FIG. 1.

FIG. 4 is an illustration of a first end view of the vacuum hand pump assembly of the embodiment of the invention depicted in FIG. 1.

FIG. 5 is an illustration of a second end view of the vacuum hand pump assembly of the embodiment of the invention depicted in FIG. 1.

FIG. 6 is an illustration of a bottom view of the vacuum hand pump assembly of the embodiment of the invention depicted in FIG. 1.

FIG. 7 is an illustration of a vacuum pump of an embodiment of the invention of the vacuum hand pump assembly of the embodiment of the invention depicted in FIG. 1.

FIG. 8 is an illustration of a base plate of the vacuum pump depicted in FIG. 7.

FIG. 9 is an illustration of a vacuum connector of the vacuum pump depicted in FIG. 7.

FIG. 10 is an illustration of a flexible vane of the vacuum pump depicted in FIG. 7.

FIG. 11 is an illustration of the flexible vane housing of the vacuum pump depicted in FIG. 7.

FIG. 12 is a perspective view of a bag valve of an embodiment of the invention for a vacuum sealable bag.

FIG. 13 is an exploded view of an embodiment of the bag valve of the invention depicted in FIG. 12.

FIG. 14 is a cross-sectional view of the bag valve depicted in FIG. 12.

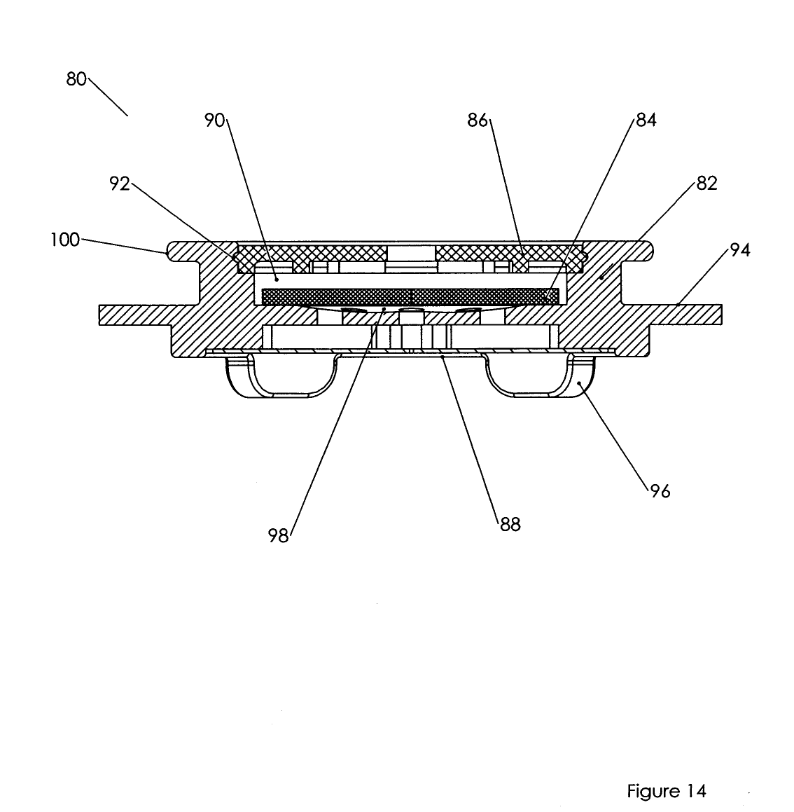

### DETAILED DESCRIPTION

Aspects of the invention are illustrated by way of example and not by way of limitation in the figures of the accompanying drawings in which like references indicate similar elements.

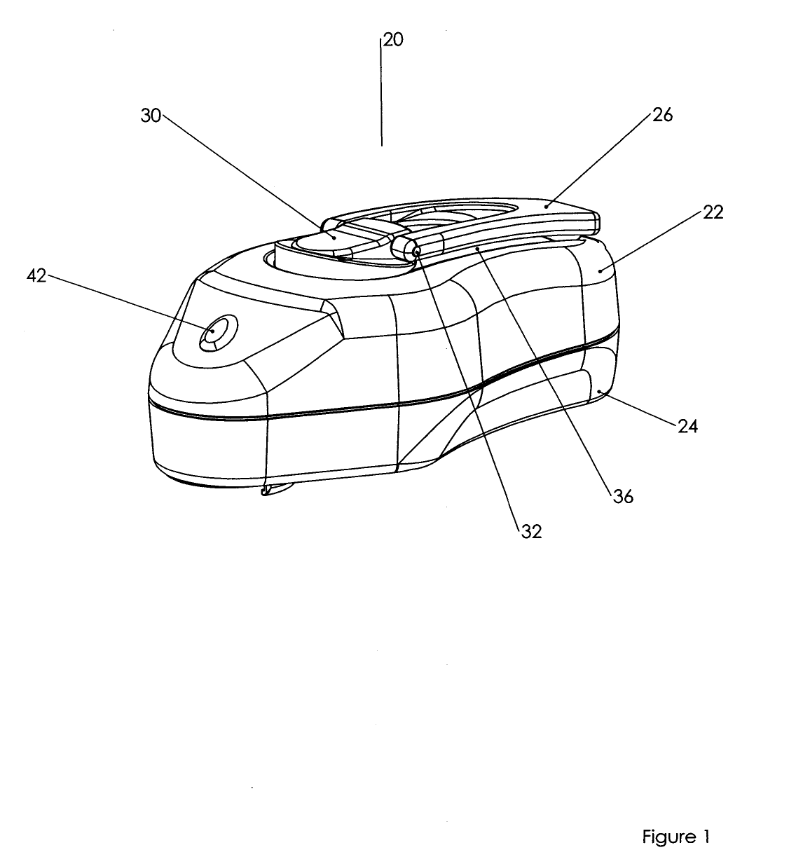

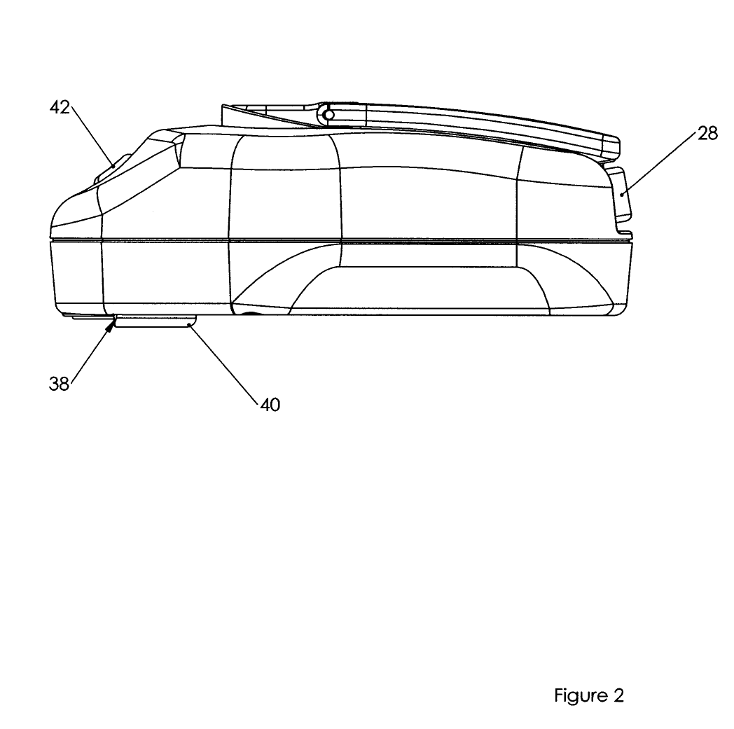

FIG. 1 is an illustration of a perspective view of a vacuum hand pump assembly 20 of an embodiment of the invention. Vacuum hand pump assembly 20 includes an upper housing shell 22 and a lower housing shell 24. Pivotally mounted on a mount associated with the pump valve (described below) and located through the upper housing shell 22 is a crank arm 26 to which a crank handle 28 is mounted (FIG. 2). Crank handle 26 is pivotally mounted on pivot mount 30. In an operating mode, crank arm 26 is pivoted about 180 degrees about the pivot axis 32 of pivot mount 30 and then rotated about the pivot mount, cranking the hand pump and generating a vacuum as will be more fully explained below.

Vacuum hand pump assembly 20 has been designed to be compact with a small footprint so that it can fit into the hand of the user, does not take up much space on a kitchen counter, and can be easily and conveniently stored in a small space. In addition, the design affords the user the ability to transport the assembly 20 to locations where it can be useful. By way of example, on a hiking, camping, or fishing trip, the assembly 20 can be conveniently packed in a backpack and used as needed to store food.

In the present embodiment the vacuum hand pump assembly is shaped much as a computer “mouse” such that it conveniently fits under at least a portion of a palm of the hand, while the other hand can turn the crank handle 28 in order to generate a vacuum. In such a position the pump assembly is held under the palm and between a person's thumb and first finger or forefinger while the crank handle is turned by the other hand. The bag valve would be positioned under the hand pump.

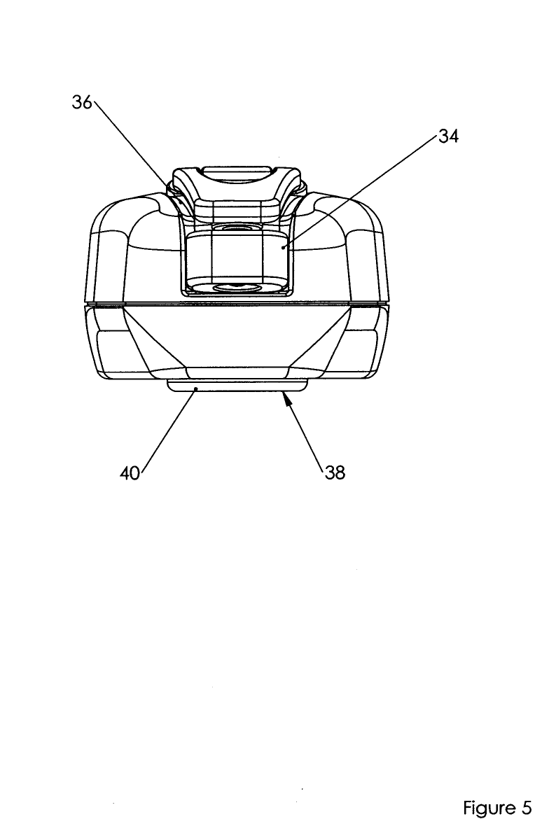

FIG. 2 is an illustration of a first side view of the vacuum hand pump assembly 20 of the embodiment of the invention. In FIG. 2, the crank handle 28 can be seen tucked in a retention groove 34 (FIG. 5) of the upper housing shell 22 of the assembly 20. The retention groove keeps the crank arm 26 and the crank handle 28 out of the way until the crank handle 28 is deployed for use to create a vacuum. In addition it is noted that there is a groove in the top surface of the upper housing shell 22 that can receive or partially receive the crank arm 26.

Extending from the bottom of the lower housing shell 24 is a vacuum mouth or vacuum interface 38 which includes a mating half ring 40 that is used to mate to the vacuum valve of a vacuum bag as described below in order to provide a vacuum to the vacuum bag. It is to be noted that the housing shells and ideally the other components of the embodiment assembly 20 can be made out of plastic parts, which are preferably molded. By way of example only the parts can be made of plastic or polyethylene or other polymer which is used for such product. As appropriate some of the parts are comprised of a rubber material or a polyurethane or a neoprene or other elastomer material.

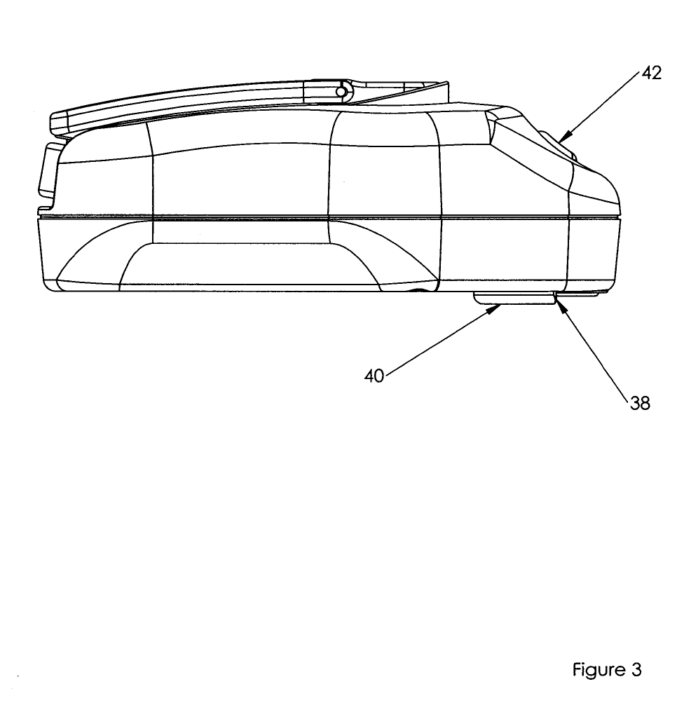

FIG. 3 is an illustration of a second side view of the vacuum hand pump assembly of the embodiment of the invention depicted in FIG. 1.

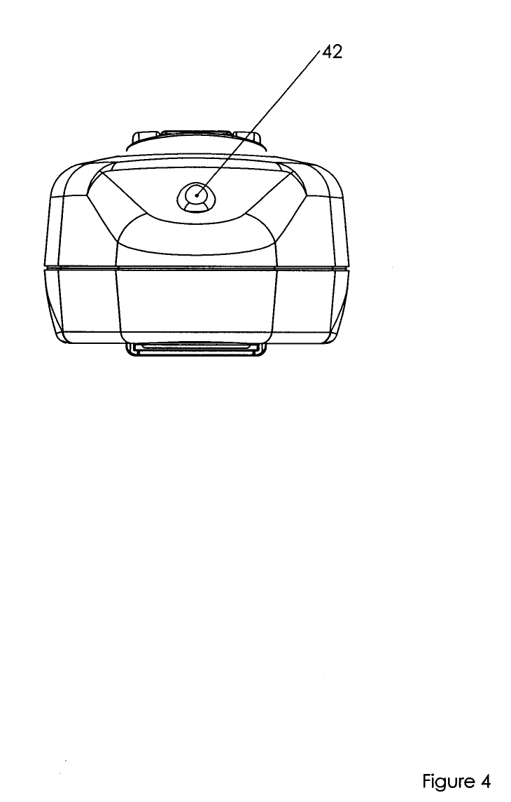

FIG. 4 is an illustration of a first end view of the vacuum hand pump assembly of the embodiment of the invention as depicted in FIG. 1. In FIG. 4 the vacuum mouth or interface 38 with the vacuum mating half ring 40 is more fully depicted. As will be explained below, an embodiment of the vacuum bag valve of the invention slides substantially parallel to the bottom of the assembly 20 and clips or snaps into the half ring 40 in order to mate with the assembly 20. In other embodiments the interface 38 can be of another design as long as a good seal is made between the vacuum interface 38 and the vacuum bag valve. By way of example only, a full ring interface can be used with the valve snapping in substantially perpendicular to the plane of the bottom of the assembly 20. FIG. 4 also depicts a button 42 which can be depressed in order to relieve the vacuum in a bag connected to the assembly 20. Depressing button 42 can open a valve in vacuum hose 70 as is known in the art. Alternatively, button 42 can be a reference point on the upper housing.

FIG. 5 is an illustration of a second end view of the vacuum hand pump assembly of the embodiment of the invention depicted in FIG. 1.

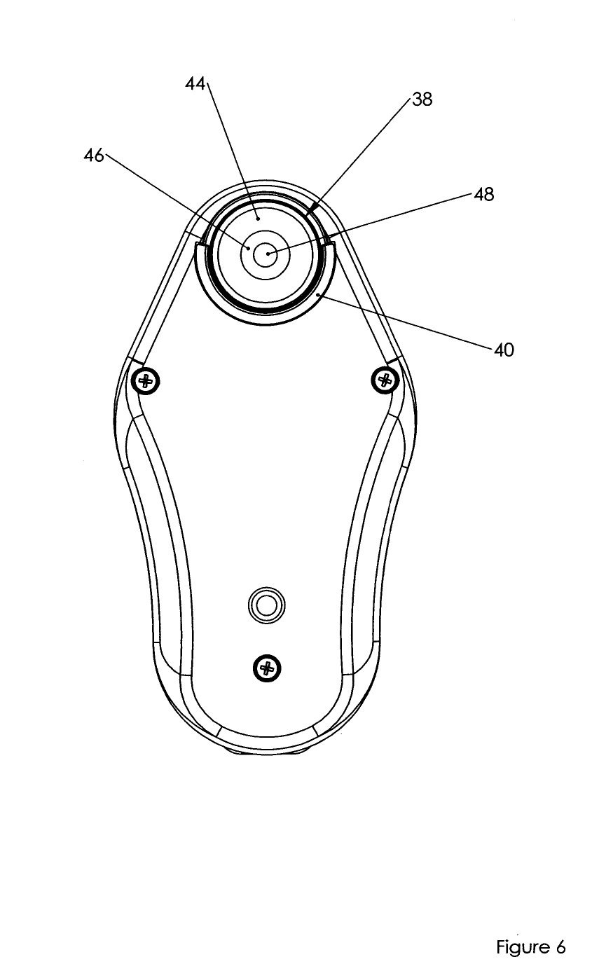

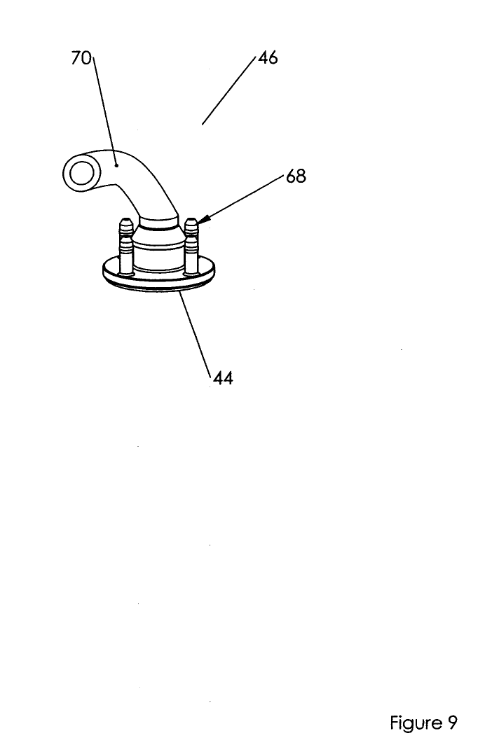

FIG. 6 is an illustration of a bottom view of the vacuum hand pump assembly of the embodiment of the invention depicted in FIG. 1. In this Figure the vacuum mouth or interface 38 and the mating ring 40 are depicted. In addition a mouth portion 44 of the vacuum connector 46 (FIG. 9) is depicted. The vacuum mouth is annular with a vacuum port 48. As will be more fully explained with respect to FIG. 9 the vacuum connector 46 is preferably made of a rubber in order to provide for the vacuum connection to the vacuum producing chamber and in order to provide an vacuum seal to the bag valve.

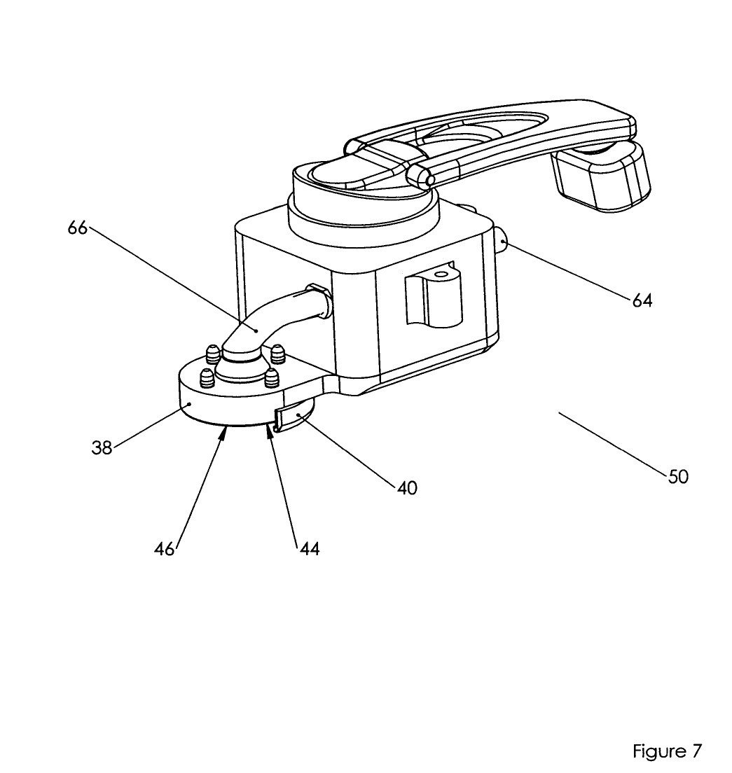

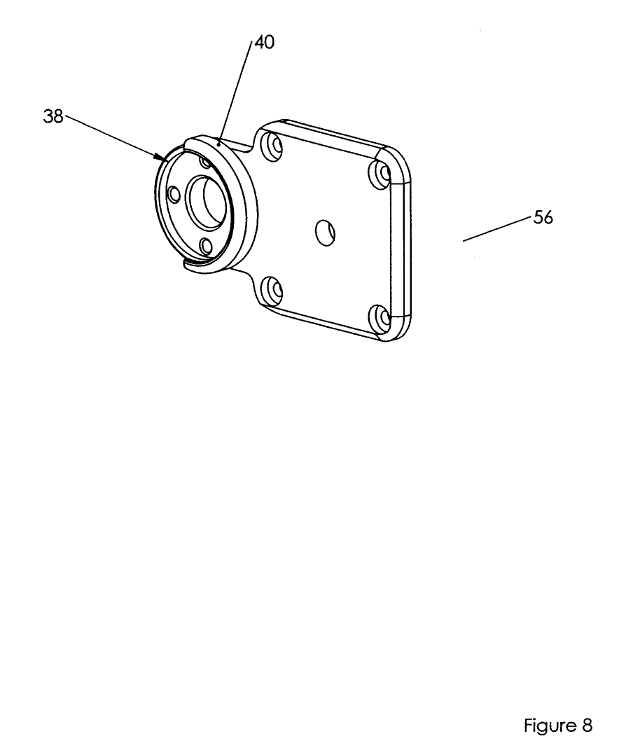

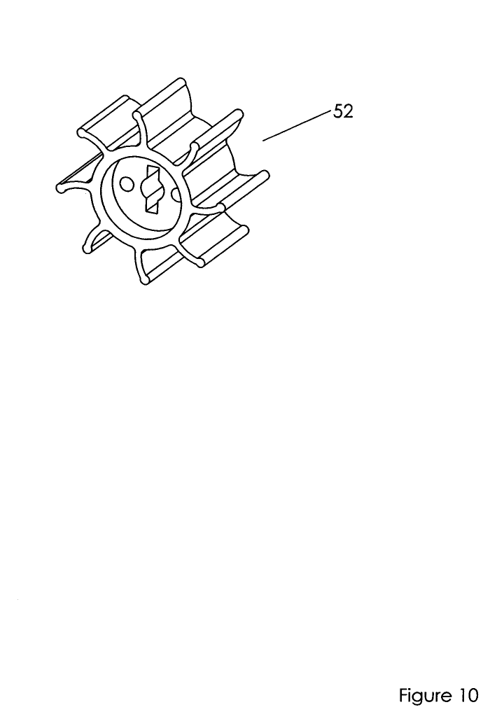

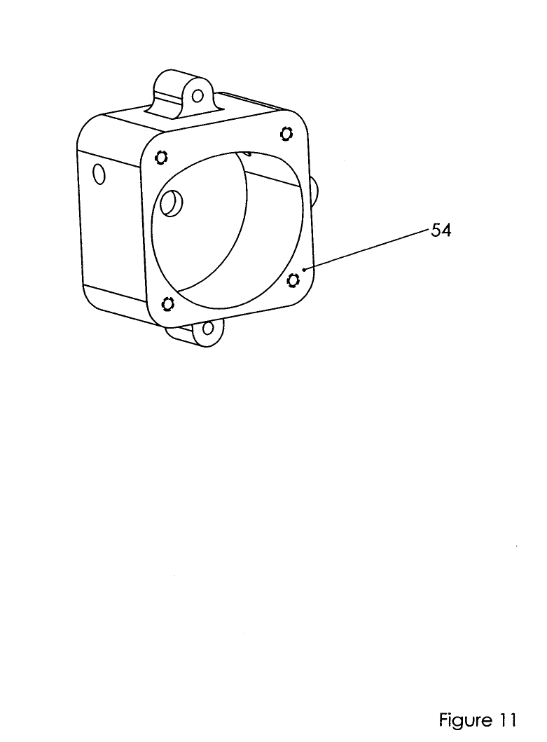

FIG. 7 is an illustration of a vacuum pump sub-assembly 50 of the vacuum pump assembly 10. The sub-assembly 50 includes a flexible vane 52 (FIG. 10), a flexible vane housing 54 (FIG. 11), a base plate 56 (FIG. 8) which is secured to the vane housing 54 in order to define a vacuum chamber 58, and the vacuum connector 46 (FIG. 9). The vacuum vane 52 is preferably comprised of a molded polyurethane or neoprene material or other elastomer material and has eight vane elements 60 extending from a central hub 62. Central hub 62 is secured to the pivot mount 30 which extends through the upper housing shell 22 (FIG. 1). The vane elements 60 are somewhat curved as they radiate from the hub 62 and end in a rounded end which can rub against the inner wall of the vane housing 54 in order to evacuate air from the vacuum chamber 58 out the outlet port 64. As can be seen in FIG. 7 the base plate 56 can be preferably bolted to the vane housing 54 in order to create the vacuum chamber 58. The connector 46 can be mounted on the vacuum interface 38. The connector 46 can be made of the same material as the vane is made of in order to create an appropriate vacuum seal. As can be seen in FIG. 9 the connector 46 has four posts 68 which can mate into the holes in the vacuum mouth or interface 38 to hold the connector 46 in place. The connector 46 includes a vacuum hose 70 which can be connected to the inlet vacuum port 66 of the vacuum chamber 58. In a preferred embodiment, the vacuum release button 42 is connected to the vacuum hose 70 with a valve. Depressing the button 42 will open the valve in the vacuum hose 70 thereby releasing any vacuum in a bag or other contained that is connected to the vacuum mount or interface 38. Alternatively, the button can be a reference point.

The vacuum hand pump assembly 20 is capable of creating a vacuum of at least three inches of mercury.

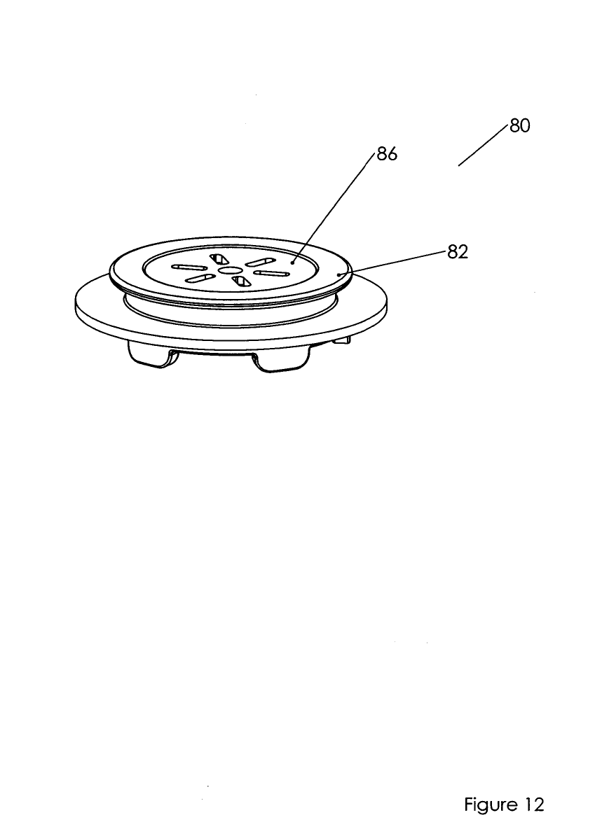

FIG. 12 is an illustration of an assembled perspective view of the bag valve 80 or an embodiment of the invention for use with a vacuum sealable bag, jar or other container that will benefit from the use of a vacuum to protect the contents stored therein.

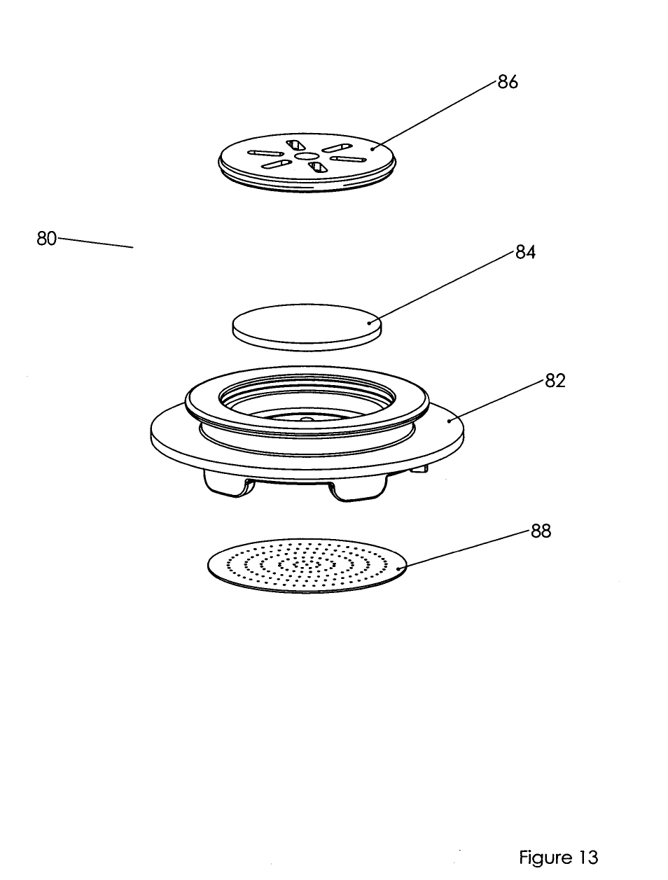

FIG. 13 is an illustration of an exploded view of the bag valve 80 as depicted in FIG. 12. In either or both of FIGS. 12 and 13 the bag valve is depicted as having a valve body or housing 82, a flexible valve element or flapper 84, a cap 86 and a filter 88. The housing 82 includes a flapper chamber 90 that received the flapper 88. The flapper chamber 90 defines an annular recess 92 into which the cap 86 can snap in order to retain the flapper 88 in the flapper chamber 90. The cap includes a series or holes or ports, deployed along radial lines from a central hole in the preferred embodiment of FIG. 13. The housing 82 includes a peripheral flange 94 that can be secured to a container such as a bag by means of heat sealing or ultrasonic bonding or in other ways known to those skilled in the art. By way of example, the valve 80 and in particular the flange 94 can be clamped down on the top of a jar with a sealing ring and used to close the jar. Extending from the housing 82 are a plurality of feet 96 that are used to keep the sides of a bag from collapsing on each other. The flapper chamber 90 includes a concave base 98 with a plurality of ports, provided therethrough, that provide vacuum communication from the valve to the bag. Preferably the flapper 84 seals about the periphery of the concave base 98 in order to create the necessary vacuum seal. The flapper is thus not required to seal directly to the ports in this preferred embodiment. In this embodiment, the valve includes a fluid filter which allows air to pass, but prevents the passage of liquids. In other embodiments, the filter is not used to prevent the passage of liquids. In this preferred embodiment, the filter can be comprised of a Gortex™ material or a TyVek™ material. Additional materials can include expanded Teflon™ or polyethylene. Such materials allow the passage of air, but prevent the passage of liquids.

As is evident from a review of the Figures and the related specification, the valve can be secured to the vacuum mouth or interface. The crank arm of the vacuum hand pump assembly can be deployed and cranked in order to create a vacuum in the container to which the valve is secure. It is noted that in a preferred embodiment that the mouth of the vacuum hand pump has been designed to optimally accept the bag valve. The half ring 40 of the mouth or interface 40 guides the engaging ring 100 of the valve until the engaging ring snaps into and is retained by the half ring 40. This arrangement is a convenient way to align the valve with the interface and obtain the seal against the mouth portion 44 of the vacuum connector 46.

The foregoing description of the preferred embodiments of the present invention has been provided for purposes of illustration and description. It is not intended to be exhaustive or to limit the invention to the precise forms disclosed. Many modifications and variations will be apparent to the practitioner skilled in the art. Embodiments were chosen and described in order to best explain the principles of the invention and its practical application, thereby enabling others skilled in the art to understand the invention, the various embodiments and with various modifications that are suited to the particular use contemplated. It is intended that the scope of the invention be defined by the following claims and their equivalents.

What is claimed is:

1. A vacuum hand pump with a deployable crank, manually rotatable vane, and a vacuum interface which is adapted to fit onto a vacuum valve.

2. The pump of claim 1 wherein the vacuum interface includes a half ring onto which a vacuum valve can be fastened.

3. A vacuum hand pump that is shaped to fit at least partially under the palm of a user's hand, with a deployable crank that can be turned by the user's other hand.

4. A vacuum valve with a flapper and a fluid barrier.

5. The valve of claim 4 wherein the flapper is confined in a chamber between a cap with ports and the fluid barrier.

6. A system for vacuum sealing a container comprising a vacuum hand pump:
said vacuum hand pump with a deployable crank, manually rotatable vane, and a vacuum interface which is adapted to fit onto a vacuum valve;
said vacuum hand pump that is shaped to fit at least partially under the palm of a user's hand, with a deployable crank that can be turned by the user's other hand; and
a vacuum valve with a flapper and a fluid barrier.

7. A vacuum hand pump and valve arrangement comprising:
a hand pump with a mouth defined by a partial ring and a sealing mouth portion;
a valve with a sealing ring;
wherein the sealing ring can be urged in a plane parallel to the half ring and guided by the half ring until the sealing ring is lockingly engaged by the partial ring.

8. The arrangement of claim 7 wherein the partial ring is a half ring.

9. A system for vacuum sealing a container comprising:
a vacuum hand pump;
said vacuum hand pump that is shaped to fit at least partially under the palm of a user's hand, with a deployable crank that can be turned by the user's other hand; and
said vacuum valve with a flapper and a fluid barrier.

10. A system for vacuum sealing a container comprising a vacuum hand pump:
said vacuum hand pump with a deployable crank, manually rotatable vane, and a vacuum interface which is adapted to fit onto a vacuum valve; and
said vacuum hand pump that is shaped to fit at least partially under the palm of a user's hand, with a deployable crank that can be turned by the user's other hand.
